SEQUENCE LISTING (9 sequences)

SEQ ID NO: 1 - DNA, 205 bp, Unknown
  Bacteria OTU1
tagatatgcggaggaacaccgatggcgaaggcaatcccctggacctgtactgacgctcatgcacgaaagcgtggggagcaaacaggattagataccctggtagtccacgccctaaacgatgtcaactggttgttgggaattagttttctcagtaacgaagctaacgcgtgaagttgaccgcctggggagtacggccgcaaggttg

SEQ ID NO: 2 - DNA, 207 bp, Unknown
  Bacteria OTU2
tagatataggaaggaacatcagtggcgaaggcggccacctggactgatactgacgctgaggtgcgaaagcgtggggagcaaacaggattagataccctggtagtccacgccgtaaacgatgtctactagccgttgggggtcttgtacctttagtggcgcagctaacgcactaagtagaccgcctggggagtacggtcgcaagattaa

SEQ ID NO: 3 - DNA, 18 bp, Artificial Sequence
  PRIMER
gtgtagcggtgraatgcg

SEQ ID NO: 4 - DNA, 41 bp, Artificial Sequence
  PRIMER
agacgtgtgctcttccgatctcccgtcaattcmtttgagtt

SEQ ID NO: 5 - DNA, 76 bp, Artificial Sequence
  PRIMER
aatgatacggcgaccaccgagatctacactctttccctacacgacgctcttccgatctgtgtagcggtgraatgcg

SEQ ID NO: 6 - DNA, 66 bp, Artificial Sequence
  PRIMER
  misc_feature (25)..(32): n is a, c, g, or t
caagcagaagacggcatacgagatnnnnnnnngtgactggagttcagacgtgtgctcttccgatct

SEQ ID NO: 7 - DNA, 293 bp, Unknown
  Bacteria OTU3
ggtgccagcagccgcggtaatacggagggtgcgagcgttaatcggaattactgggcgtaaagcgcatgcaggtggtttgttaagtcagatgtgaaagcccggggctcaacctcggaatagcatttgaaactggcagactagagtactgtagaggggggtagaatttcaggtgtagcggtgaaatgcgtagagatctgaaggaataccggtggcgaaggcggccccctggacagatactgacactcagatgcgaaagcgtggggagcaaacaggattagaaacccctgtagtcc

SEQ ID NO: 8 - DNA, 293 bp, Unknown
  Bacteria OTU4
ggtgccagcagccgcggtaatacggagggtgcgagcgttaatcggaattactgggcgtaaagcgcatgcaggtggtttgttaagtcagatgtgaaagcccggggctcaacctcggaatagcatttgaaactggcagactagagtactgtagaggggggtagaatttcaggtgtagcggtgaaatgcgtagagatctgaaggaataccggtggcgaaggcggccccctggacagatactgacactcagatgcgaaagcgtggggagcaaacaggattagatacccctgtagtcc

SEQ ID NO: 9 - DNA, 293 bp, Unknown
  Bacteria OTU5
ggtgccagcagccgcggtaatacggagggtgcaagcgttaatcggaattactgggcgtaaagcgcgcgtaggcggccaagtcagtcagatgtgaaagccccgggcttaacctgggaactgcacctgatactgcttggctagagtacagaagagggtggtggaatttcctgtgtagcggtgaaatgcgtagatataggaaggaacatcagtggcgaaggcggccacctggtctgatactgacgctgaggtgcgaaagcgtggggagcaaacaggattagaaacccctgtagtcc

(19) United States
(12) Patent Application Publication
(10) Pub. No.: US 2023/0212696 A1
(43) Pub. Date: Jul. 6, 2023

(54) FISH SKIN MICROBIOME

(71) Applicants: The State of Israel , Ministry of Agriculture & Rural Development, Agricultural Research Organizatio, Rishon LeZion (IL); ISRAEL OCEANOGRAPHIC AND LIMNOLOGICAL RESEARCH, LTD., Eilat (IL); The Dead-Sea and Arava Science Center (DSASC), Masada National Park (IL)

(72) Inventors: Galit SHARON, Be'er Ora (IL); Ashraf Mohammad Fahmi Rashad AL ASHHAB, East Jerusalem (IL)

(73) Assignees: The State of Israel , Ministry of Agriculture & Rural Development, Agricultural Research Organizatio, Rishon LeZion (IL); ISRAEL OCEANOGRAPHIC AND LIMNOLOGICAL RESEARCH, LTD., Eilat (IL); The Dead-Sea and Arava Science Center (DSASC), Masada National Park (IL)

(21) Appl. No.: 17/638,190

(22) PCT Filed: Aug. 25, 2020
(86) PCT No.: PCT/IB2020/057943

§ 371 Date: Feb. 25, 2022

Related U.S. Application Data
(60) Provisional application No. 62/891,437, filed on Aug. 26, 2019.

(51) Int. Cl. C12Q 1/689; A61K 35/741; A61P 31/04

(52) U.S. Cl. CPC C12Q 1/689; A61K 35/741; A61P 31/04; A61K 2035/115

(57) ABSTRACT

The present disclosure provides method and compositions for predicting and increasing the survival of fish after stress.

### CROSS REFERENCE TO RELATED APPLICATION

This application is a National Phase Application of PCT International Application No. PCT/IB2020/057943, International Filing Date Aug. 25, 2020, claiming the benefit of U.S. Patent Application No. 62/891,437, filed Aug. 26, 2019 which is hereby incorporated by reference.

### SEQUENCE LISTING STATEMENT

The instant application contains a Sequence Listing which has been submitted electronically in ASCII format and is hereby incorporated by reference in its entirety. Said ASCII copy, created on Nov. 14, 2022, is named P-583939-US-SQL-ST25-14NOV22.txt and is 3.7 KB in size.

### BACKGROUND OF THE DISCLOSURE

Increased fish consumption, population growth and fish' great contribution to food security and social development; have made fish aquaculture to show the fastest growing food sector globally with an average of 8% annual increase over the last 30 years. This increase in production has been accompanied with an increase in known diseases and the development of new diseases as a result of increased stress conditions and reduced immunity fish are facing in intensive growth conditions.

Stress in fish can be broadly defined as a state in which a series of adaptive responses re-establish homeostasis following exposure to a stressor. In fish, the stress response includes activation of the hypothalamus-pituitary-interrenal (HPI) axis, culminating in the release of glucocorticoids from internal cells located in the head kidney. In intensive aquaculture, farmed fish are frequently exposed to stressors such as crowding and handling, which can impact health and welfare, and threaten aquaculture sustainability. In the wild as well, natural fish populations are increasingly subject to multiple anthropogenic stressors which threaten their sustainability. In particular, stress-mediated impairment of immune function has been widely described in cultured and wild fish, and associated with an increased susceptibility to disease.

In fish, mucosal immune response plays a crucial role in the course of the infection and includes a healthy and dynamic microbial communities. In this context, recent studies have found fish skin microbiome to play an important role in fish health during infection, stress condition and antibiotic applications.

There remains a need in the field of commercial aquaculture of fish for products and methods to monitor, prevent, ameliorate and treat pathological microbial infections, which have devastating monetary effects on farmers.

### SUMMARY OF THE DISCLOSURE

The present disclosure is based on the results of advanced methodologies identifying a link between changes in the microbial communities' present in the skin of healthy fish, and in different stages of disease and recovery.

The present disclosure provides, in one aspect, a method of predicting the survival of a fish after stress, comprising testing the fish for the presence of a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

The present disclosure provides, in another aspect, a method of increasing the survival of a fish after a stress, comprising administering to the fish a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

The present disclosure provides, in yet another aspect, a composition comprising a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the method comprises testing the fish for the presence of bacteria comprising a 16S gene comprising the nucleotide sequence set forth in SEQ ID NO: 1. In certain embodiments, the method comprises testing the fish for the presence of bacteria comprising a 16S gene comprising the nucleotide sequence set forth in SEQ ID NO: 2. In certain embodiments, the method comprises testing the fish for the presence of bacteria comprising a 16S gene comprising the nucleotide sequence set forth in SEQ ID NO: 7. In certain embodiments, the method comprises testing the fish for the presence of bacteria comprising a 16S gene comprising the nucleotide sequence set forth in SEQ ID NO: 8. In certain embodiments, the method comprises testing the fish for the presence of bacteria comprising a 16S gene comprising the nucleotide sequence set forth in SEQ ID NO: 9.

In certain embodiments, the method comprises testing the fish for the presence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the fish is of the Class Actinopterygii. In certain embodiments, the fish is of the Order Perciformes.

In certain embodiments, the fish is of the Family Sparidae. In certain embodiments, the fish is of the Genus Sparus. In certain embodiments, the fish is Sparus aurata.

In certain embodiments, the fish is of the Family Latidae. In certain embodiments, the fish is of the Genus Lates. In certain embodiments, the fish is Lates calcarifer.

In certain embodiments, the stress is selected from the group consisting of infection with pathogenic bacteria, netting stress, physical injury, and any combination thereof.

In certain embodiments, the stress is infection with pathogenic bacteria. In certain embodiments, the stress is netting stress. In certain embodiments, the stress is physical injury. In certain embodiments, the stress is mild physical injury. In certain embodiments, the stress is local physical injury. In certain embodiments, the stress is mild local physical injury. In certain embodiments, the stress is a scratch. In certain embodiments, the stress is a local scratch. In certain embodiments, the stress is a mild scratch. In certain embodiments, the stress is a needle scratch. In certain embodiments, the stress is descaling. In certain embodiments, the stress is local descaling. In certain embodiments, the stress is mild descaling.

In certain embodiments, the stress is infection with pathogenic bacteria, netting stress, and physical injury. In certain embodiments, the stress is infection with pathogenic bacteria, netting stress, and a scratch. In certain embodiments, the stress is infection with pathogenic bacteria, netting stress, and descaling.

In certain embodiments, the pathogenic bacteria are Gram-negative bacteria.

In certain embodiments, the Gram-negative bacteria are selected from the group consisting of the Genus Vibrio, the Genus Pseudomonas, the Genus Edwardsiella, and the Genus Mycobacterium.

In certain embodiments, the Gram-negative bacteria are of the Genus Vibrio. In certain embodiments, the Gram-negative bacteria are of the Genus Pseudomonas. In certain embodiments, the Gram-negative bacteria are of the Genus Edwardsiella. In certain embodiments, the Gram-negative bacteria are of the Genus Mycobacterium.

In certain embodiments, the pathogenic bacteria are Vibrio harveyi.

In certain embodiments, the pathogenic bacteria are Gram-positive bacteria.

In certain embodiments, the Gram-positive bacteria are selected from the group consisting of the Genus Streptococcus, and the Genus Lactococcus.

In certain embodiments, the Gram-positive bacteria are of the Genus Streptococcus. In certain embodiments, the Gram-positive bacteria are of the Genus Lactococcus.

In certain embodiments, the pathogenic bacteria are Streptococcus iniae.

In certain embodiments, the presence of a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof, is predictive of survival of the fish after the stress.

In certain embodiments, the presence of a bacterium comprising a 16S gene comprising the nucleotide sequence set forth in SEQ ID NO: 1 is predictive of survival of the fish after the stress. In certain embodiments, the presence of a bacterium comprising a 16S gene comprising the nucleotide sequence set forth in SEQ ID NO: 2 is predictive of survival of the fish after the stress. In certain embodiments, the presence of a bacterium comprising a 16S gene comprising the nucleotide sequence set forth in SEQ ID NO: 7 is predictive of survival of the fish after the stress. In certain embodiments, the presence of a bacterium comprising a 16S gene comprising the nucleotide sequence set forth in SEQ ID NO: 8 is predictive of survival of the fish after the stress. In certain embodiments, the presence of a bacterium comprising a 16S gene comprising the nucleotide sequence set forth in SEQ ID NO: 9 is predictive of survival of the fish after the stress.

In certain embodiments, the presence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of survival of the fish after the stress

In certain embodiments, the absence of a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof, is predictive of death of the fish after the stress.

In certain embodiments, the absence of a bacterium comprising a 16S gene comprising the nucleotide sequence set forth in SEQ ID NO: 1 is predictive of death of the fish after the stress. In certain embodiments, the absence of a bacterium comprising a 16S gene comprising the nucleotide sequence set forth in SEQ ID NO: 2 is predictive of death of the fish after the stress. In certain embodiments, the absence of a bacterium comprising a 16S gene comprising the nucleotide sequence set forth in SEQ ID NO: 7 is predictive of death of the fish after the stress. In certain embodiments, the absence of a bacterium comprising a 16S gene comprising the nucleotide sequence set forth in SEQ ID NO: 8 is predictive of death of the fish after the stress. In certain embodiments, the absence of a bacterium comprising a 16S gene comprising the nucleotide sequence set forth in SEQ ID NO: 9 is predictive of death of the fish after the stress.

In certain embodiments, the absence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of death of the fish after the stress.

In certain embodiments, the method further comprises administering to the fish a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the method further comprises administering to the fish a bacterium comprising a 16S gene comprising the nucleotide sequence set forth in SEQ ID NO: 1. In certain embodiments, the method further comprises administering to the fish a bacterium comprising a 16S gene comprising the nucleotide sequence set forth in SEQ ID NO: 2. In certain embodiments, the method further comprises administering to the fish a bacterium comprising a 16S gene comprising the nucleotide sequence set forth in SEQ ID NO: 7. In certain embodiments, the method further comprises administering to the fish a bacterium comprising a 16S gene comprising the nucleotide sequence set forth in SEQ ID NO: 8. In certain embodiments, the method further comprises administering to the fish a bacterium comprising a 16S gene comprising the nucleotide sequence set forth in SEQ ID NO: 9.

In certain embodiments, the method further comprises administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the method of increasing the survival of a fish after a stress, comprises administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the composition comprises bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the composition is for use in a method of increasing the survival of a fish after a stress, comprising administering to the fish a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the composition is for use in a method of increasing the survival of a fish after a stress, comprising administering to the fish a bacterium comprising a 16S gene comprising the nucleotide sequence set forth in SEQ ID NO: 1. In certain embodiments, the composition is for use in a method of increasing the survival of a fish after a stress, comprising administering to the fish a bacterium comprising a 16S gene comprising the nucleotide sequence set forth in SEQ ID NO: 2. In certain embodiments, the composition is for use in a method of increasing the survival of a fish after a stress, comprising administering to the fish a bacterium comprising a 16S gene comprising the nucleotide sequence set forth in SEQ ID NO: 7. In certain embodiments, the composition is for use in a method of increasing the survival of a fish after a stress, comprising administering to the fish a bacterium comprising a 16S gene comprising the nucleotide sequence set forth in SEQ ID NO: 8. In certain embodiments, the composition is for use in a method of increasing the survival of a fish after a stress, comprising administering to the fish a bacterium comprising a 16S gene comprising the nucleotide sequence set forth in SEQ ID NO: 9.

In certain embodiments, the composition is for use in a method of increasing the survival of a fish after a stress, comprising administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

These and other aspects and embodiments of the present disclosure are disclosed in the following Detailed Description, the appended Claims, and the accompanying Figures.

### BRIEF DESCRIPTION OF THE DRAWINGS

FIG. 1. The y-axis shows the bacterial phylum relative abundance of fish skin microbial communities found in (A) UV-treated water in winter (water temperature 19° C. to 23° C.); (B) UV-untreated water in winter and (C) UV-untreated water in summer (water temperature 24° C. to 28° C.) experiments. The x-axis shows the different time interval of sampling point at T0, T1, T2 and T3, corresponding to control condition before Vibrio harveyi infection (T0), 72 h after infection at stress and disease condition (T1), one week after infection (recovery stage) (T2), and three weeks after the infection (later recovery stage) (T3), respectively.

FIG. 2. The abundance of each of the main two OTU's throughout the experiment.

FIG. 3. Phylogenetic analysis of unknown OTU1 showing 97.1% sequence similarity with closely related stains.

FIG. 4. Phylogenetic analysis of unknown OTU2 showing 100% sequence similarity with closely related uncultured stains.

FIG. 5. Survival rate of both Lates calcarifer and Sparus aurata fish species in UV-treated and UV-untreated water, up to 14 days post infection by immersion with Gram-positive Streptococcus iniae.

FIG. 6. The total relative abundance of different species phyla (color) and highlighted species of Streptococcus iniae (Gram-positive pathogen) in red and Vibrionaceae spp. in dark blue in different individual fish for both UV-treated and UV-untreated water for the two fish species at different time points.

FIG. 7. Streptococcaceae family relative abundance at different time points for Lates calcarifer (FIG. 7A) and Sparus aurata (FIG. 7B). The letter on top of each bar resembles statistical significance>0.05.

FIG. 8. Vibrionaceae family relative abundance at different time points for Lates calcarifer (FIG. 8A) and Sparus aurata (FIG. 8B). The letter on top of each bar resembles statistical significance>0.05.

FIG. 9. Phylogenetic tree analysis showing the related species compared to unclassified OTU's.

FIG. 10. Sequence classification based on RDP blast search with sequences similarity to closely related sequences in database.

### DETAILED DESCRIPTION OF THE DISCLOSURE

The disclosure characterizes the variability in skin bacterial composition in healthy and diseased fish, such as Gilthead Seabream (Sparus aurata) and Barramundi (Lates calcarifer). Based on the surprising experimental findings presented herein, modifications and balance in fish skin flora (in sick and health conditions) are better understood, and are harnessed for providing beneficial tools for e.g. commercial fish farmers. Methods and compositions are provided, to predict fish response to potentially-deadly stress, and to prevent disease and increase fish health and welfare. Importantly, these new tools to manipulate fish skin flora and control microbiome composition enhance commercial fish yields.

In addition to the methods provided in the present disclosure, five different bacteria, herein labeled OTUs 1, 2, 3, 4 and 5, are now isolated and characterized for the first time. As exemplified herein, these bacteria are beneficially utilized to predict fish survival, prevent fish death, and treat fish after stress.

The present disclosure provides, in one aspect, a method of predicting the survival of a fish after stress, comprising testing the fish for the presence of a bacterium of the Class Betaproteobacteria or of the Class Gammaproteobacteria.

The present disclosure provides, in another aspect, a method of increasing the survival of a fish after a stress, comprising administering to the fish a bacterium of the Class Betaproteobacteria or of the Class Gammaproteobacteria.

The present disclosure provides, in yet another aspect, a composition comprising a bacterium of the Class Betaproteobacteria or of the Class Gammaproteobacteria.

In certain embodiments, the bacteria of the Class Betaproteobacteria is of the Order Burkholderiales. In certain embodiments, the bacteria of the Order Burkholderiales is of the Family Comamonadaceae. In certain embodiments, the bacteria of the Family Comamonadaceae is of the Genus Delftia. In certain embodiments, the bacteria of the Genus Delftia is comprising a 16S gene comprising the nucleotide sequence set forth in SEQ ID NO: 1.

In certain embodiments, bacteria of the Class Gammaproteobacteria is of the Order Oceanospirillales. In certain embodiments, bacteria of the Order Oceanospirillales is of the Family Oceanospirillaceae. In certain embodiments, bacteria of the Family Oceanospirillaceae is of the Genus Profundimonas. In certain embodiments, bacteria of the Genus Profundimonas is comprising a 16S gene comprising the nucleotide sequence set forth in SEQ ID NO: 2.

In certain embodiments, bacteria of the Class Gammaproteobacteria is of the Order Vibrionales. In certain embodiments, bacteria of the Order Vibrionales is of the Family Vibrionaceae. In certain embodiments, bacteria of the Family Vibrionaceae is of the Genus Catenococcus. In certain embodiments, bacteria of the Genus Catenococcus is comprising a 16S gene comprising the nucleotide sequence set forth in SEQ ID NO: 7.

In certain embodiments, bacteria of the Class Gammaproteobacteria is of the Order Vibrionales. In certain embodiments, bacteria of the Order Vibrionales is of the Family Vibrionaceae. In certain embodiments, bacteria of the Family Vibrionaceae is of the Genus Catenococcus. In certain embodiments, bacteria of the Genus Catenococcus is comprising a 16S gene comprising the nucleotide sequence set forth in SEQ ID NO: 8.

In certain embodiments, bacteria of the Class Gammaproteobacteria is of the Order Oceanospirillales. In certain embodiments, bacteria of the Order Oceanospirillales is of the Family Oceanospirillaceae. In certain embodiments, bacteria of the Family Oceanospirillaceae is of the Genus Profundimonas. In certain embodiments, bacteria of the Genus Profundimonas is comprising a 16S gene comprising the nucleotide sequence set forth in SEQ ID NO: 9.

The present disclosure provides, in one aspect, a method of predicting the survival of a fish after stress, comprising testing the fish for the presence of a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

The present disclosure provides, in another aspect, a method of increasing the survival of a fish after a stress, comprising administering to the fish a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

The present disclosure provides, in yet another aspect, a composition comprising a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

A person of skill in the art would understand that, as exemplified herein, testing fish for the presence of a bacterium, or of bacteria in general, includes taking a sample of bacteria from e.g. the outer surface of the skin of the fish, and checking the 16S ribosomal RNA gene of the bacteria for a nucleotide sequence. Methods for both steps are well known in the art.

A person of skill in the art would further know that 16S ribosomal RNA (or 16S rRNA) is the component of the 30S small subunit of a prokaryotic ribosome, and the genes coding for it are used in reconstructing phylogenies, due to the slow rate of evolution of this region of the gene.

It should be understood that testing one fish includes testing of a plurality of fish, that checking one gene includes testing of a plurality of genes, and that looking for a nucleotide sequence includes looking for a plurality of nucleotide sequences. It should further be understood that different steps can be performed simultaneously or sequentially.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

A person of skill in the field would understand that administering bacteria to fish can be done in a variety of methods and steps, as long known in the field. For example, bacteria may be e.g. directly administered to the outer, skin surface of the fish, or alternatively, bacteria may be e.g. indirectly administered to the fish by adding the bacteria to the water surrounding the fish. In addition, bacteria may be administered to the inner surface of the fish e.g. by feeding the fish with such bacteria.

In certain embodiments, the bacterium comprising a 16S gene comprising the nucleotide sequence set forth in SEQ ID NO: 1 is administered in water temperature of 24° C. to 28° C. In certain embodiments, the bacterium comprising a 16S gene comprising the nucleotide sequence set forth in SEQ ID NO: 1 is administered in water temperature of 25° C. to 27° C. In certain embodiments, the bacterium comprising a 16S gene comprising the nucleotide sequence set forth in SEQ ID NO: 1 is administered in water temperature of 24° C. In certain embodiments, the bacterium comprising a 16S gene comprising the nucleotide sequence set forth in SEQ ID NO: 1 is administered in water temperature of 25° C. In certain embodiments, the bacterium comprising a 16S gene comprising the nucleotide sequence set forth in SEQ ID NO: 1 is administered in water temperature of 26° C. In certain embodiments, the bacterium comprising a 16S gene comprising the nucleotide sequence set forth in SEQ ID NO: 1 is administered in water temperature of 27° C. In certain embodiments, the bacterium comprising a 16S gene comprising the nucleotide sequence set forth in SEQ ID NO: 1 is administered in water temperature of 28° C.

In certain embodiments, the bacterium comprising a 16S gene comprising the nucleotide sequence set forth in SEQ ID NO: 2 is administered in water temperature of 19° C. to 23° C. In certain embodiments, the bacterium comprising a 16S gene comprising the nucleotide sequence set forth in SEQ ID NO: 2 is administered in water temperature of 20° C. to 21° C. In certain embodiments, the bacterium comprising a 16S gene comprising the nucleotide sequence set forth in SEQ ID NO: 2 is administered in water temperature of 19° C. In certain embodiments, the bacterium comprising a 16S gene comprising the nucleotide sequence set forth in SEQ ID NO: 2 is administered in water temperature of 20° C. In certain embodiments, the bacterium comprising a 16S gene comprising the nucleotide sequence set forth in SEQ ID NO: 2 is administered in water temperature of 21° C. In certain embodiments, the bacterium comprising a 16S gene comprising the nucleotide sequence set forth in SEQ ID NO: 2 is administered in water temperature of 22° C. In certain embodiments, the bacterium comprising a 16S gene comprising the nucleotide sequence set forth in SEQ ID NO: 2 is administered in water temperature of 23° C.

In certain embodiments, the bacterium comprising a 16S gene comprising the nucleotide sequence set forth in SEQ ID NO: 1 is administered in water temperature of 24° C. to 28° C. and the bacterium comprising a 16S gene comprising the nucleotide sequence set forth in SEQ ID NO: 2 is administered in water temperature of 19° C. to 23° C. In certain embodiments, the bacterium comprising a 16S gene comprising the nucleotide sequence set forth in SEQ ID NO: 1 is administered in water temperature of 25° C. to 27° C. and the bacterium comprising a 16S gene comprising the nucleotide sequence set forth in SEQ ID NO: 2 is administered in water temperature of 20° C. to 22° C.

In certain embodiments, the method comprises testing the fish for the presence of bacteria comprising a 16S gene comprising the nucleotide sequence set forth in SEQ ID NO: 1. In certain embodiments, the method comprises testing the fish for the presence of bacteria comprising a 16S gene comprising the nucleotide sequence set forth in SEQ ID NO: 2. In certain embodiments, the method comprises testing the fish for the presence of bacteria comprising a 16S gene comprising the nucleotide sequence set forth in SEQ ID NO: 7. In certain embodiments, the method comprises testing the fish for the presence of bacteria comprising a 16S gene comprising the nucleotide sequence set forth in SEQ ID NO: 8. In certain embodiments, the method comprises testing the fish for the presence of bacteria comprising a 16S gene comprising the nucleotide sequence set forth in SEQ ID NO: 9.

In certain embodiments, the method comprises testing the fish for the presence of bacteria comprising a 16S gene comprising the nucleotide sequence set forth in at least two of SEQ ID NO: 1, SEQ ID NO: 2, SEQ ID NO: 7, SEQ ID NO: 8, and SEQ ID NO: 9.

In certain embodiments, the method comprises testing the fish for the presence of bacteria comprising a 16S gene comprising the nucleotide sequence set forth in at least three of SEQ ID NO: 1, SEQ ID NO: 2, SEQ ID NO: 7, SEQ ID NO: 8, and SEQ ID NO: 9.

In certain embodiments, the method comprises testing the fish for the presence of bacteria comprising a 16S gene comprising the nucleotide sequence set forth in at least four of SEQ ID NO: 1, SEQ ID NO: 2, SEQ ID NO: 7, SEQ ID NO: 8, and SEQ ID NO: 9.

In certain embodiments, the method comprises testing the fish for the presence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the fish is of the Class Actinopterygii. In certain embodiments, the fish is of the Order Perciformes.

In certain embodiments, the fish is of the Family Sparidae. In certain embodiments, the fish is of the Genus Sparus. In certain embodiments, the fish is Sparus aurata.

In certain embodiments, the fish is of the Family Latidae. In certain embodiments, the fish is of the Genus Lates. In certain embodiments, the fish is Lates calcarifer.

In certain embodiments, the fish is of the Subclass Chondrostei. In certain embodiments, the fish is of the Subclass Neopterygii. In certain embodiments, the fish is of the Subclass Cladistia.

A person of skill in the art would appreciate that physical, chemical and perceived stressors can separately evoke responses in fish, which are considered adaptive to enable the fish to cope with the disturbance and maintain its homeostatic state. In certain embodiments, the stress evokes a response in the fish. In certain embodiments, the stress comprises activation of the hypothalamus-pituitary-interrenal (HPI) axis. In certain embodiments, the stress comprises release of glucocorticoids from internal cells located in the head kidney. In certain embodiments, the stress comprises activation of the hypothalamus-pituitary-internal (HPI) axis and release of glucocorticoids from internal cells located in the head kidney.

In certain embodiments, the stress is selected from the group consisting of infection with pathogenic bacteria, netting stress, physical injury, and any combination thereof. In certain embodiments, the stress is selected from the group consisting of infection with pathogenic bacteria, netting stress, and physical injury.

In certain embodiments, the stress is infection with pathogenic bacteria. In certain embodiments, the stress is netting stress. In certain embodiments, the stress is physical injury. In certain embodiments, the stress is mild physical injury. In certain embodiments, the stress is local physical injury. In certain embodiments, the stress is mild local physical injury. In certain embodiments, the stress is a scratch. In certain embodiments, the stress is a local scratch. In certain embodiments, the stress is a mild scratch. In certain embodiments, the stress is a needle scratch. In certain embodiments, the stress is descaling. In certain embodiments, the stress is local descaling. In certain embodiments, the stress is mild descaling.

In certain embodiments, the stress is at least two of infection with pathogenic bacteria, netting stress, and physical injury.

The term “pathogenic bacteria” as used herein refers to any disease-causing bacteria. In certain embodiments, the pathogenic bacteria cause disease in fish. In certain embodiments, the pathogenic bacteria cause disease in Sparus aurate fish. In certain embodiments, the pathogenic bacteria cause disease in Lates calcarifer fish.

In certain embodiments, the stress is infection with pathogenic bacteria, netting stress, and physical injury. In certain embodiments, the stress is infection with pathogenic bacteria, netting stress, and a scratch. In certain embodiments, the stress is infection with pathogenic bacteria, netting stress, and descaling.

A person of skill in the art would understand that netting stress is a form of physical handling which causes stress to fish as they are unable to move freely and/or unable to breath properly, and that physical injury cause stress to fish as they are no longer physically intact.

In certain embodiments, the pathogenic bacteria are Gram-negative bacteria.

In certain embodiments, the Gram-negative bacteria are selected from the group consisting of the Genus Vibrio, the Genus Pseudomonas, the Genus Edwardsiella, and the Genus Mycobacterium.

In certain embodiments, the Gram-negative bacteria are of the Genus Vibrio. In certain embodiments, the Gram-negative bacteria are of the Genus Pseudomonas. In certain embodiments, the Gram-negative bacteria are of the Genus Edwardsiella. In certain embodiments, the Gram-negative bacteria are of the Genus Mycobacterium.

In certain embodiments, the pathogenic bacteria are Vibrio harveyi.

In certain embodiments, the pathogenic bacteria are Gram-positive bacteria.

In certain embodiments, the Gram-positive bacteria are selected from the group consisting of the Genus Streptococcus, and the Genus Lactococcus.

In certain embodiments, the Gram-positive bacteria are of the Genus Streptococcus. In certain embodiments, the Gram-positive bacteria are of the Genus Lactococcus.

In certain embodiments, the pathogenic bacteria are Streptococcus iniae.

In certain embodiments, the presence of a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof, is predictive of survival of the fish after the stress.

In certain embodiments, the presence of a bacterium comprising a 16S gene comprising the nucleotide sequence set forth in SEQ ID NO: 1 is predictive of survival of the fish after the stress. In certain embodiments, the presence of a bacterium comprising a 16S gene comprising the nucleotide sequence set forth in SEQ ID NO: 2 is predictive of survival of the fish after the stress. In certain embodiments, the presence of a bacterium comprising a 16S gene comprising the nucleotide sequence set forth in SEQ ID NO: 7 is predictive of survival of the fish after the stress. In certain embodiments, the presence of a bacterium comprising a 16S gene comprising the nucleotide sequence set forth in SEQ ID NO: 8 is predictive of survival of the fish after the stress. In certain embodiments, the presence of a bacterium comprising a 16S gene comprising the nucleotide sequence set forth in SEQ ID NO: 9 is predictive of survival of the fish after the stress.

In certain embodiments, the presence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of survival of the fish after the stress.

In certain embodiments, the survival is in a water temperature of 24° C. to 28° C. In certain embodiments, the survival is in a water temperature of 25° C. to 27° C. In certain embodiments, the survival is in a water temperature of 24° C. In certain embodiments, the survival is in a water temperature of 25° C. In certain embodiments, the survival is in a water temperature of 26° C. In certain embodiments, the survival is in a water temperature of 27° C. In certain embodiments, the survival is in a water temperature of 28° C.

In certain embodiments, the survival is in a water temperature of 19° C. to 23° C. In certain embodiments, the survival is in a water temperature of 20° C. to 22° C. In certain embodiments, the survival is in a water temperature of 19° C. In certain embodiments, the survival is in a water temperature of 20° C. In certain embodiments, the survival is in a water temperature of 21° C. In certain embodiments, the survival is in a water temperature of 22° C. In certain embodiments, the survival is in a water temperature of 23° C.

In certain embodiments, the presence of a bacterium comprising a 16S gene comprising the nucleotide sequence set forth in SEQ ID NO: 1 and of a bacterium comprising a 16S gene comprising the nucleotide sequence set forth in SEQ ID NO: 2 is predictive of survival of the fish after the stress. In certain embodiments, the survival is in a water temperature of 19° C. to 28° C. In certain embodiments, the survival is in a water temperature of 20° C. to 27° C. In certain embodiments, the survival is in a water temperature of 21° C. to 26° C. In certain embodiments, the survival is in a water temperature of 22° C. to 25° C. In certain embodiments, the survival is in a water temperature of 23° C. to 24° C. In certain embodiments, the survival is in a water temperature of 19° C. In certain embodiments, the survival is in a water temperature of 20° C. In certain embodiments, the survival is in a water temperature of 21° C. In certain embodiments, the survival is in a water temperature of 22° C. In certain embodiments, the survival is in a water temperature of 23° C. In certain embodiments, the survival is in a water temperature of 24° C. In certain embodiments, the survival is in a water temperature of 25° C. In certain embodiments, the survival is in a water temperature of 26° C. In certain embodiments, the survival is in a water temperature of 27° C. In certain embodiments, the survival is in a water temperature of 28° C.

In certain embodiments, the absence of a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof, is predictive of death of the fish after the stress.

In certain embodiments, the absence of a bacterium comprising a 16S gene comprising the nucleotide sequence set forth in SEQ ID NO: 1 is predictive of death of the fish after the stress. In certain embodiments, the absence of a bacterium comprising a 16S gene comprising the nucleotide sequence set forth in SEQ ID NO: 2 is predictive of death of the fish after the stress. In certain embodiments, the absence of a bacterium comprising a 16S gene comprising the nucleotide sequence set forth in SEQ ID NO: 7 is predictive of death of the fish after the stress. In certain embodiments, the absence of a bacterium comprising a 16S gene comprising the nucleotide sequence set forth in SEQ ID NO: 8 is predictive of death of the fish after the stress. In certain embodiments, the absence of a bacterium comprising a 16S gene comprising the nucleotide sequence set forth in SEQ ID NO: 9 is predictive of death of the fish after the stress.

In certain embodiments, the absence of a bacterium comprising a 16S gene comprising the nucleotide sequence set forth in SEQ ID NO: 1 is predictive of death of the fish in water temperature of 24° C. to 28° C. after the stress. In certain embodiments, the absence of a bacterium comprising a 16S gene comprising the nucleotide sequence set forth in SEQ ID NO: 1 is predictive of death of the fish in water temperature of 25° C. to 27° C. after the stress. In certain embodiments, the absence of a bacterium comprising a 16S gene comprising the nucleotide sequence set forth in SEQ ID NO: 1 is predictive of death of the fish in water temperature of 26° C. after the stress.

In certain embodiments, the absence of a bacterium comprising a 16S gene comprising the nucleotide sequence set forth in SEQ ID NO: 2 is predictive of death of the fish in water temperature of 19° C. to 23° C. after the stress. In certain embodiments, the absence of a bacterium comprising a 16S gene comprising the nucleotide sequence set forth in SEQ ID NO: 2 is predictive of death of the fish in water temperature of 20° C. to 22° C. after the stress. In certain embodiments, the absence of a bacterium comprising a 16S gene comprising the nucleotide sequence set forth in SEQ ID NO: 2 is predictive of death of the fish in water temperature of 21° C. after the stress.

In certain embodiments, the absence of a bacterium comprising a 16S gene comprising the nucleotide sequence set forth in SEQ ID NO: 1 and the absence of a bacterium comprising a 16S gene comprising the nucleotide sequence set forth in SEQ ID NO: 2 is predictive of death of the fish after the stress. In certain embodiments, the death is in a water temperature of 19° C. to 28° C. In certain embodiments, the death is in a water temperature of 20° C. to 27° C. In certain embodiments, the death is in a water temperature of 21° C. to 26° C. In certain embodiments, the death is in a water temperature of 22° C. to 25° C. In certain embodiments, the death is in a water temperature of 23° C. to 24° C. In certain embodiments, the death is in a water temperature of 19° C. In certain embodiments, the death is in a water temperature of 20° C. In certain embodiments, the death is in a water temperature of 21° C. In certain embodiments, the death is in a water temperature of 22° C. In certain embodiments, the death is in a water temperature of 23° C. In certain embodiments, the death is in a water temperature of 24° C. In certain embodiments, the death is in a water temperature of 25° C. In certain embodiments, the death is in a water temperature of 26° C. In certain embodiments, the death is in a water temperature of 27° C. In certain embodiments, the death is in a water temperature of 28° C.

In certain embodiments, the absence of a bacterium comprising a 16S gene comprising the nucleotide sequence set forth in at least two of SEQ ID NO: 1, SEQ ID NO: 2, SEQ ID NO: 7, SEQ ID NO: 8, or SEQ ID NO: 9, is predictive of death of the fish after the stress. In certain embodiments, the absence of a bacterium comprising a 16S gene comprising the nucleotide sequence set forth in at least three of SEQ ID NO: 1, SEQ ID NO: 2, SEQ ID NO: 7, SEQ ID NO: 8, or SEQ ID NO: 9, is predictive of death of the fish after the stress. In certain embodiments, the absence of a bacterium comprising a 16S gene comprising the nucleotide sequence set forth in at least four of SEQ ID NO: 1, SEQ ID NO: 2, SEQ ID NO: 7, SEQ ID NO: 8, or SEQ ID NO: 9, is predictive of death of the fish after the stress.

In certain embodiments, the absence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of death of the fish after the stress.

In certain embodiments, the absence of a bacterium comprising a 16S gene comprising the nucleotide sequence set forth in SEQ ID NO: 1 is predictive of death of the fish in water temperature of 24° C. to 28° C. after the stress. In certain embodiments, the absence of a bacterium comprising a 16S gene comprising the nucleotide sequence set forth in SEQ ID NO: 2 is predictive of death of the fish in water temperature of 19° C. to 23° C. after the stress. In certain embodiments, the absence of a bacterium comprising a 16S gene comprising the nucleotide sequence set forth in SEQ ID NO: 1 and the absence of a bacterium comprising a 16S gene comprising the nucleotide sequence set forth in SEQ ID NO: 2 is predictive of death of the fish after the stress. In certain embodiments, the death is in a water temperature of 19° C. to 28° C. In certain embodiments, the bacterium comprising a 16S gene comprising the nucleotide sequence set forth in SEQ ID NO: 1 is administered in water temperature of 24° C. to 28° C. In certain embodiments, the bacterium comprising a 16S gene comprising the nucleotide sequence set forth in SEQ ID NO: 2 is administered in water temperature of 19° C. to 23° C. In certain embodiments, the bacterium comprising a 16S gene comprising the nucleotide sequence set forth in SEQ ID NO: 1 is administered in water temperature of 24° C. to 28° C. and the bacterium comprising a 16S gene comprising the nucleotide sequence set forth in SEQ ID NO: 2 is administered in water temperature of 19° C. to 23° C.

In certain embodiments, the method further comprises administering to the fish a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the method further comprises administering to the fish a bacterium comprising a 16S gene comprising the nucleotide sequence set forth in SEQ ID NO: 1. In certain embodiments, the method further comprises administering to the fish a bacterium comprising a 16S gene comprising the nucleotide sequence set forth in SEQ ID NO: 2. In certain embodiments, the method further comprises administering to the fish a bacterium comprising a 16S gene comprising the nucleotide sequence set forth in SEQ ID NO: 7. In certain embodiments, the method further comprises administering to the fish a bacterium comprising a 16S gene comprising the nucleotide sequence set forth in SEQ ID NO: 8. In certain embodiments, the method further comprises administering to the fish a bacterium comprising a 16S gene comprising the nucleotide sequence set forth in SEQ ID NO: 9.

In certain embodiments, the method further comprises administering to the fish a bacterium comprising a 16S gene comprising the nucleotide sequence set forth in at least two of SEQ ID NO: 1, SEQ ID NO: 2, SEQ ID NO: 7, SEQ ID NO: 8, or SEQ ID NO: 9. In certain embodiments, the method further comprises administering to the fish a bacterium comprising a 16S gene comprising the nucleotide sequence set forth in at least three of SEQ ID NO: 1, SEQ ID NO: 2, SEQ ID NO: 7, SEQ ID NO: 8, or SEQ ID NO: 9. In certain embodiments, the method further comprises administering to the fish a bacterium comprising a 16S gene comprising the nucleotide sequence set forth in at least four of SEQ ID NO: 1, SEQ ID NO: 2, SEQ ID NO: 7, SEQ ID NO: 8, or SEQ ID NO: 9.

In certain embodiments, the method further comprises administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the method of increasing the survival of a fish after a stress, comprises administering to the fish bacteria comprising a 16S gene comprising the nucleotide sequence set forth in at least two of SEQ ID NO: 1, SEQ ID NO: 2, SEQ ID NO: 7, SEQ ID NO: 8, and SEQ ID NO: 9. In certain embodiments, the method of increasing the survival of a fish after a stress, comprises administering to the fish bacteria comprising a 16S gene comprising the nucleotide sequence set forth in at least three of SEQ ID NO: 1, SEQ ID NO: 2, SEQ ID NO: 7, SEQ ID NO: 8, and SEQ ID NO: 9. In certain embodiments, the method of increasing the survival of a fish after a stress, comprises administering to the fish bacteria comprising a 16S gene comprising the nucleotide sequence set forth in at least four of SEQ ID NO: 1, SEQ ID NO: 2, SEQ ID NO: 7, SEQ ID NO: 8, and SEQ ID NO: 9.

In certain embodiments, the method of increasing the survival of a fish after a stress, comprises administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the composition comprises a plurality of a bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the plurality of the bacterium comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9, or of any combination thereof, is at least 10%, is at least 20%, is at least 30%, is at least 40%, is at least 50%, is at least 60%, is at least 70%, is at least 80%, or is at least 90%, by weight of the total weight of the composition.

In certain embodiments, the composition further comprises water. In certain embodiments, the composition is substantially devoid of water. In certain embodiments, the composition further comprises salt. In certain embodiments, the composition further comprises at least 5% by weight salt. In certain embodiments, the composition further comprises 3.5% to 4.5% by weight salt. In certain embodiments, the composition comprises less than 3% by weight salt. In certain embodiments, the composition comprises less than 2% by weight salt. In certain embodiments, the composition comprises less than 1% by weight salt. In certain embodiments, the composition is substantially devoid of salt.

In certain embodiments, the composition further comprises a carrier. In certain embodiments, the composition further comprises a synthetic carrier. The term “synthetic” as used herein includes “unnatural”, “not found in nature”, “man-made” and “machine-made”.

In certain embodiments, the composition is a veterinary composition. In certain embodiments, the composition further comprises a veterinary excipient. In certain embodiments, the composition is a pharmaceutical composition. In certain embodiments, the composition further comprises a pharmaceutical excipient. A person of skill in the art would understand the terms “veterinary composition”, “veterinary excipient”, “pharmaceutical composition”, and “pharmaceutical excipient” to be of high purity and commercial standards.

The term “pharmaceutical excipient” as used herein refers to any excipient used in a pharmaceutical, food or veterinary product. It may be an excipient having the function of diluent, binder, coating, non-stick, disintegrating, fluidizing, solubilizing, lubricant, stabilizer, anti-caking, anti-moisture, taste masking or load, modification of the release profile (extended release, delayed release, etc.) etc. The term is further intended to mean any therapeutically inactive substance used as a carrier for the active ingredients of a medication. The term “pharmaceutical excipient” is used herein in its common technical meaning and refers to all substances other than the active ingredient which are included in a ready-for-use pharmaceutical preparation.

In certain embodiments, the composition comprises bacteria comprising a 16S gene comprising the nucleotide sequence set forth in at least two of SEQ ID NO: 1, SEQ ID NO: 2, SEQ ID NO: 7, SEQ ID NO: 8, and SEQ ID NO: 9. In certain embodiments, the composition comprises bacteria comprising a 16S gene comprising the nucleotide sequence set forth in at least three of SEQ ID NO: 1, SEQ ID NO: 2, SEQ ID NO: 7, SEQ ID NO: 8, and SEQ ID NO: 9. In certain embodiments, the composition comprises bacteria comprising a 16S gene comprising the nucleotide sequence set forth in at least four of SEQ ID NO: 1, SEQ ID NO: 2, SEQ ID NO: 7, SEQ ID NO: 8, and SEQ ID NO: 9.

In certain embodiments, the composition comprises bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the composition is for use in a method of increasing the survival of a fish after a stress, comprising administering to the fish a bacterium comprising a 16S gene comprising the nucleotide sequence set forth in SEQ ID NO: 1, SEQ ID NO: 2, SEQ ID NO: 7, SEQ ID NO: 8, or SEQ ID NO: 9, or of any combination thereof.

In certain embodiments, the composition is for use in a method of increasing the survival of a fish after a stress, comprising administering to the fish a bacterium comprising a 16S gene comprising the nucleotide sequence set forth in SEQ ID NO: 1. In certain embodiments, the composition is for use in a method of increasing the survival of a fish after a stress, comprising administering to the fish a bacterium comprising a 16S gene comprising the nucleotide sequence set forth in SEQ ID NO: 2. In certain embodiments, the composition is for use in a method of increasing the survival of a fish after a stress, comprising administering to the fish a bacterium comprising a 16S gene comprising the nucleotide sequence set forth in SEQ ID NO: 7. In certain embodiments, the composition is for use in a method of increasing the survival of a fish after a stress, comprising administering to the fish a bacterium comprising a 16S gene comprising the nucleotide sequence set forth in SEQ ID NO: 8. In certain embodiments, the composition is for use in a method of increasing the survival of a fish after a stress, comprising administering to the fish a bacterium comprising a 16S gene comprising the nucleotide sequence set forth in SEQ ID NO: 9.

In certain embodiments, the composition is for use in a method of increasing the survival of a fish after a stress, comprising administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

In certain embodiments, the phrase “increasing the survival of a fish after a stress” means that more fish would survive a stress after being administered with one or more of the bacteria provided herein compared to the same fish after the same stress without being administered with the one or more of the bacteria provided herein.

In certain embodiments, bacteria comprising a 16S gene comprising the nucleotide sequence set forth in SEQ ID NO: 1 is of the Class Betaproteobacteria, or of the Order Burkholderiales, or of the Family Comamonadaceae, or of the Genus Delftia.

In certain embodiments, bacteria comprising a 16S gene comprising the nucleotide sequence set forth in SEQ ID NO: 2 is of the Class Gammaproteobacteria, or of the Order Oceanospirillales, or of the Family Oceanospirillaceae, or of the Genus Profundimonas.

In certain embodiments, bacteria comprising a 16S gene comprising the nucleotide sequence set forth in SEQ ID NO: 7 is of the Class Gammaproteobacteria, or of the Order Vibrionales, or of the Family Vibrionaceae, or of the Genus Catenococcus.

In certain embodiments, bacteria comprising a 16S gene comprising the nucleotide sequence set forth in SEQ ID NO: 8 is of the Class Gammaproteobacteria, or of the Order Vibrionales, or of the Family Vibrionaceae, or of the Genus Catenococcus.

In certain embodiments, bacteria comprising a 16S gene comprising the nucleotide sequence set forth in SEQ ID NO: 9 is of the Class Gammaproteobacteria, or of the Order Oceanospirillales, or of the Family Oceanospirillaceae, or of the Genus Profundimonas.

### EXAMPLES

### Example 1

The following experiment was designed to examine the role of microbiome in fish immunity and survival, the fish skin microbiome was compared in (i) UV-treated water and (ii) UV-untreated water during winter and summer seasons. The fish skin microbiome was sampled at different locations including abdomen, later line, gills and anus of Sparus aurata (Gilt-head bream, also known as “Dennis”) at (i) control conditions, (ii) stress condition and disease condition, (iii) recovery period, and (iv) three weeks after recovery.

At each experiment (summer and winter), two 50 L fish tank were placed next to each other in a stabilized environment at a growth facility. Each fish tank contained 10 fish of Sparus aurata that were marked with a P-tag. Both tanks were under a continuous flow of (i) UV-treated and (ii) UV-untreated seawater, fish in both tanks were fed once a day. Before starting each experiment, fish were reared in each separate tank for to be acclimated to the environment.

Both stress and disease conditions were introduced to fish in both tank as following: first, fish were subjected to netting stress when fish were placed in a net outside of the water for 7 minutes. Following the netting stress, fish were subjected to mild physical injury (local needle scratch and/or local descaling) at their tails and placed back in their relative tank. At the same time, tank water volume was reduced to 20 L and water flow was stopped. Next, Vibrio harveyi was added to fish tanks at 250,000 bacteria/ml and an immersion period of the pathogenic Vibrio harveyi was allowed by keeping the tank volume to 20 L for 1 h. Afterward, the water flow in each tank returned to its initial state and fish were monitored throughout the experiments.

Fish skin microbial communities were collected from each individual using a sterile cotton swab as following: fish from each tank were taken out of their tank and placed on a sterile tray (20×50 cm). Then, cotton swabs were rapped on ˜1 cm² area on fish skin at the area of their later line and placed on ice. At the same time inserted P-tags were read and were marked on the swab sample. Following swab collection, fish were placed back in an intermediate tank until all fish in the tank were sampled and placed back immediately in their original fish tank after sample collection.

Two identical experiments were carried out in the winter (January) and in the summer (September). At each experiment, fish swab samples were collected in both tanks at initial condition of (i) control (TO) and (ii) stress and disease condition 24 h after infection (T1). For those fish who survived, additional samples were collected at the (iii) recovery period (T2) and (iv) three weeks after recovery (T3), as illustrated in Table 1.

TABLE 1
Swab sample collection throughout the experiments.

| | | | Total number of fish | |
|---|---|---|---|---|
| Season | Tank | Time point | sampled | Date |
| Winter | UV-treated | Control (T0) | 7 out of 7 | Jan 11 |
| Winter | UV-treated | Sick (T1) | 7 out of 7 | Jan 14 |
| Winter | UV- | Control (T0) | 8 out of 8 | Jan 11 |
| | untreated | | | |
| Winter | UV- | Sick (T1) | 8 out of 8 | Jan 14 |
| | untreated | | | |
| Winter | UV- | Recovery | 2 out of 2 survived | Jan 18 |
| | untreated | (T2) | | |
| Winter | UV- | Recovery | 2 out of 2 survived | Jan 27 |
| | untreated | (T3) | | |
| Summer | UV-treated | Control (T0) | 10 out of 10 | Sep 4 (not |
| | | | | analyzed) |
| Summer | UV-treated | Sick (T1) | 10 out of 10 | Sep 7 (not |
| | | | | analyzed) |
| Summer | UV- | Control (T0) | 10 out of 10 | Sep 4 |
| | untreated | | | |
| Summer | UV- | Sick (T1) | 10 out of 10 | Sep 7 |
| | untreated | | | |
| Summer | UV- | Recovery | 4 out of 4 survived | Sep 13 |
| | untreated | (T2) | | |
| Summer | UV- | Recovery | 4 out of 4 survived | Sep 27 |
| | untreated | (T3) | | |

DNA extraction: Swab samples taken from different treatments and time points were individually clipped under sterile conditions and placed for DNA extraction using MoBio 96 well plate Soil DNA extraction Kit, following the manufacturer's protocol. All steps of DNA extraction were carried out in a sterile UV-hood to reduce external contamination. In every DNA extraction 96 well plate, DNA extraction negative control were added by placing 200 μl of RNase free water and all samples were placed randomly in the DNA extraction plate to exclude any bias.

PCR and library preparation: PCR using modified 16s rDNA gene were used to amplify 16S rDNA gene (Table 2). All PCR reactions were performed in triplicates where each replicate was performed in a separate 96-well plate. The PCR reactions were prepared by mixing 10 μl of ready-mixed KAPA HIFI, 0.4 μl of equal v/v mixed primers, 7.6 μl of RNase free water and 2 μl of DNA template, as following: 98° C.; 2 min, 35 cycles of; 98° C.; 10 s, 61° C.; 15 s, 72° C.; 35 s and 72° C.; 5 min for extension and then holding at 4° C.

TABLE 2
The position of each primer,
sequence and length of each 16S DNA
amplicon used in the First PCR protocol.

| | Sequence | Forward | Reverse | |
|---|---|---|---|---|
| | (5′-3′) | primer | Primer | Product |
| Primer | [Concentration] | position | position | length |
| F649 | Forward primer: | 649 | 889 | 240 |
| | GTGTAGCGGTGRAAT | | | |
| | GCG | | | |
| | [SEQ ID NO: 3] | | | |
| | [20 μM] | | | |
| R889 | Reverse primer: | | | |
| | AGACGTGTGCTCTTC | | | |
| | CGATCTCCCGTCAAT | | | |
| | TCMTTTGAGTT | | | |
| | [20 μM] | | | |
| | [SEQ ID NO: 4] | | | |

After the first PCR, samples were run on 1.5% agarose gel electrophoresis to confirm the required band were successfully amplified with no amplification in the negative controls, then all PCR triplicates were pooled together. Upon pooling, a PCR cleaning step was performed to all the samples to get rid of all primers and nucleotides by mixing 36 μl of Agencourt® AMPure® XP BECKMAN COULTER bead solution to 45 μl of each of the pooled DNA (recommended ratio for less than 200 bp fragment exclusion based on manufacturer protocol). After adding the bead solution, samples were mixed well by pipetting few times and incubated at room temperature for 5 min. After incubation, samples were placed on a magnetic stand for 2 minutes to remove the beads with the desired fragments (more than 200 bp) and supernatant was discarded. Then, magnetic beads were washed twice with freshly prepared 200 μl of 80% ethanol and 30 s incubation time between two washes, then left 10 min for air-dry. Upon drying, 43 μl of DDW with 10 mM Tris (pH=8.5) were added to each sample with 10 times pipette mixing, incubated at room temperature for 2 min, allowing the supernatant to clear, and 42 μl of the supernatant were aliquoted in different PCR sterile tubes and stored in −80° C. for second PCR and library preparation.

Library preparation was performed using a second PCR to connect the illumina linker, adapter and unique 8 base pair barcode to each sample (Table 3). The second PCR reactions were prepared by mixing 21 μl of ready-mixed KAPA HIFI, 2 μl of mixed forward primers, 12.6 μl of RNase free water to al 4 μl of each sample from the first PCR product with 2 μl of barcoded reverse primer, and placed in thermocycler at the following conditions; 98° C.; 2 min, 8 cycles of; 98° C.; 10 s, 64° C.; 15 s, 72° C.; 25 s and 72° C.; 5 min for extension and then hold at 4° C. Upon finishing, all PCR product were pooled together and subjected to cleaning as previously mentioned in the first PCR cleaning, however 50 μl of pooled second PCR product were cleaned using 1:1 ratio with the bead solution for more conservative size exclusion of fragments less than 200 bp, and at the final step, 50 μl of DDW with 10 mM Tris (pH=8.5) were added to each sample and 48 μl of the supernatant were aliquoted to sterile PCR tubes, saved in −80° C. and 15 μl of the final product were sent to PE 300 Miseq Illumina sequencing, such that each lane consisted of 96 samples.

TABLE 3
The position of each primer, sequence
and length of each 16S DNA amplicon
used in the second PCR, library
preparation protocol. N₈-different
nucleotide sequences used as a barcode
to tag the different samples in the
process of library preparation.

| Pair # | Sequence (5′-3′) [Concentration] |
|---|---|
| Forward | AATGATACGGCGACCACCGAGATCTACACTCT |
| primer | TTCCCTACACGACGCTCTTCCGATCTGTGTAG |
| | CGGTGRAATGCG [8 μM] |
| | [SEQ ID NO: 5] |
| Reverse | CAAGCAGAAGACGGCATACGAGATN₈GTGACT |
| primer | GGAGTTCAGACGTGTGCTCTTCCGATCT |
| | [8 μM] [SEQ ID NO: 6] |

Sequence analysis and quality control: At first, a series of sequence checks and quality control were performed on the analysis as following; (i) both paired-end were merged using PEAR software, then sequences were cleaned for low quality score, ambiguous bases, chimeric sequences and PCR errors using “qiime dada2 denoise-paired” at qiime2 Software. Then, all sequences were clustered at 0.99 sequences similarity using “vsearch cluster-features-open-reference”and classified using the “feature-classifier classify-sklearn” open reference 16S rRNA based Silva V13.8 data base. Then, the obtained operational taxonomic units (OTU) table was cleaned for sequences classified as D_0_mitochondria, D_0_Archaea, Unassigned, D_4_Mitochondria, D_3_Chloroplast and D_0_Eukaryota.

Assessment of community composition: From the obtained Qiime2 classified OTU table, R statistical and analysis software were used to generate the relative abundances of microbial communities. At first, rare abundant OTUs were removed from the OTU table, a cut off 5,000 of row sums of all samples for a single OTU were removed. Many cutoffs including 500, 1000, 2000 and 5000 cutoff were tested and noticed no significant changes in the microbial community's compositions at lower cutoffs. 5,000 total sequences number per OTU were chosen as a cutoff. Afterward, the OTU table were normalized by total sum of each sample, and a bar graph was generated. In bar graph each color were assigned to those OTUs classified under same phylum with one exception for those OTUs showed a high abundance of more than 25% of the total at any sampling stage, thus a different color for those OTUs were assigned and illustrated under the same bar graph.

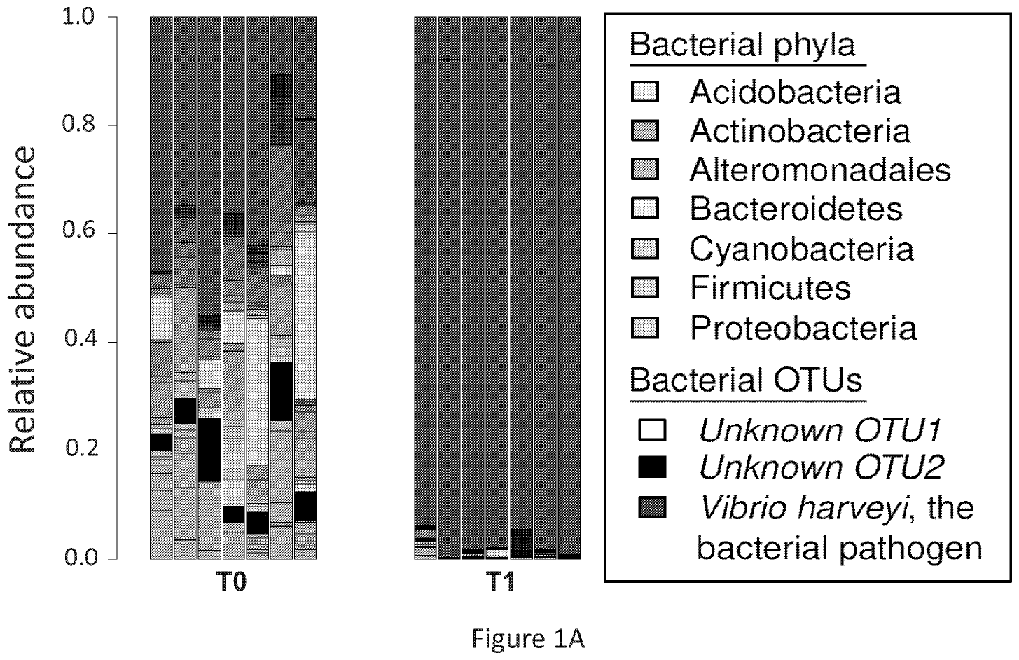

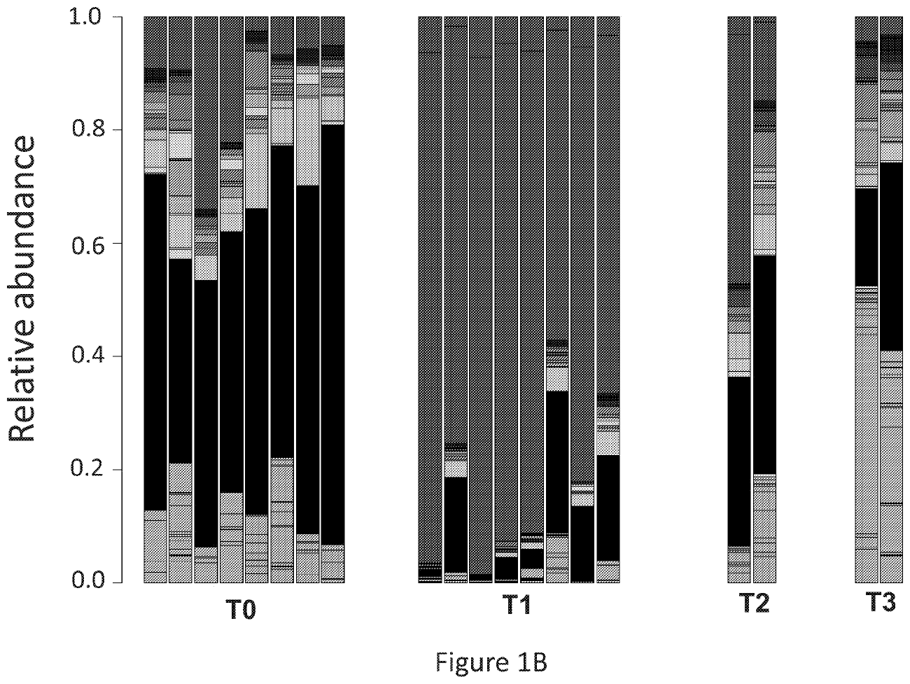

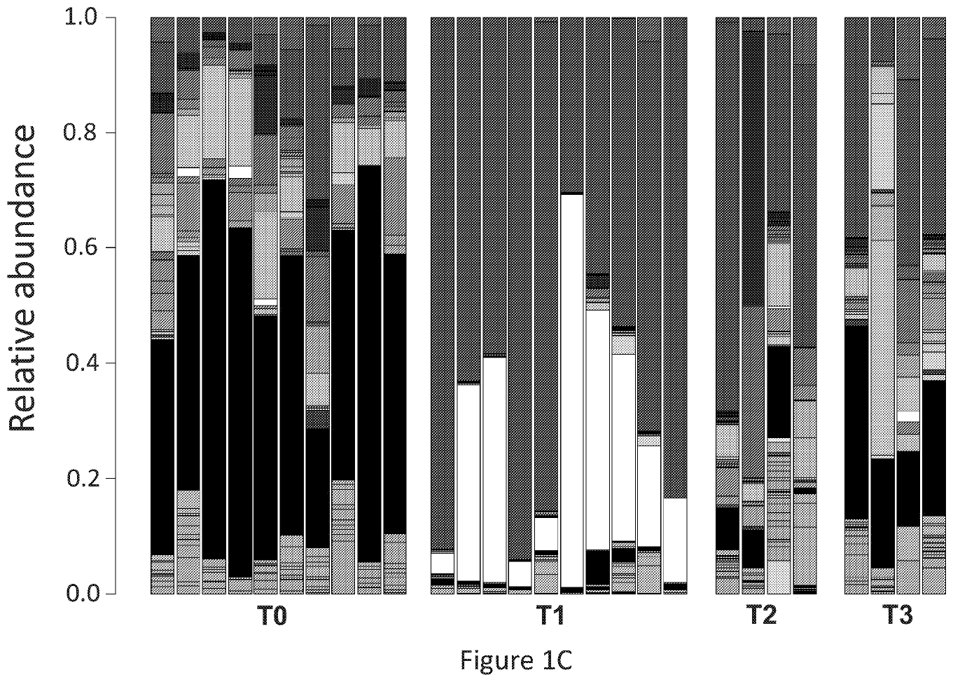

A total of 62 samples were collected for both UV treated and UV-untreated seawater fish skin microbial communities with an average row sequences number of 36,304 sequences and an average of 32,252 of cleaned non-chimeric sequences per each data set were obtained. These sequences were then classified and a bar graph showing the relative abundances of each taxonomic unit is presented in FIG. 1.

As presented in FIG. 1, those fish reared in a UV-untreated seawater, showed a 40% survival rate compared to those reared in UV-treated water (presented in T2 and T3) following a controlled infection with Vibrio harveyi. The bar graph of each plot is placed in synchronized order of fish tag, meaning those survived fish in T2 and T3 are corresponding to the last bars in T0 and T1. In both FIGS. 1B and 1C there is no clear or significant abundant of any OTU's or bacterial phyla comparing those survived fish over those who didn't. However, a clear and significant abundance of two main OTU's were present when comparing fish skin microbial communities in fishes grown in UV-untreated seawater (FIG. 1B and FIG. 1C) to those grown in UV-treated seawater.

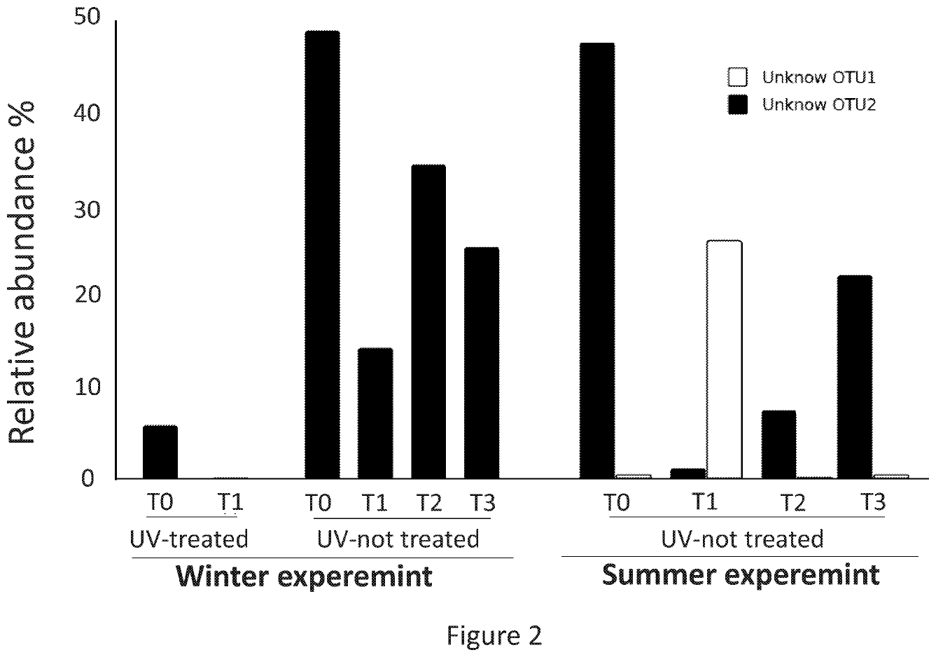

Interestingly, one of those OTUs, unknown OTU2 (TAGATATAGGAAGGAACATCAGTGGCGAAGGCGGCCACCTGGACTGATACTGACG CTGAGGTGCGAAAGCGTGGGGAGCAAACAGGATTAGATACCCTGGTAGTCCACGCC GTAAACGATGTCTACTAGCCGTTGGGGGTCTTGTACCTTTAGTGGCGCAGCTAACGC ACTAAGTAGACCGCCTGGGGAGTACGGTCGCAAGATTAA) [SEQ ID NO: 2] were abundant in both summer and winter experiment at T0 but showed a higher abundance at disease conditions at T1 only for those fish grown in winter (FIG. 2).

On the other hand, the unknown OTU1 (TAGATATGCGGAGGAACACCGATGGCGAAGGCAATCCCCTGGACCTGTACTGACGC TCATGCACGAAAGCGTGGGGAGCAAACAGGATTAGATACCCTGGTAGTCCACGCCC TAAACGATGTCAACTGGTTGTTGGGAATTAGTTTTCTCAGTAACGAAGCTAACGCGT GAAGTTGACCGCCTGGGGAGTACGGCCGCAAGGTTG) [SEQ ID NO: 1] showed to have the highest abundance at T1 during the summer experiment. Interestingly, there is a slight abundance of the unknown OTU1 at T0 for summer experiment which was absent at winter experiment (FIG. 2 and Table 4).

TABLE 4
The percent abundance of each of the two main
OTU's in each experiment and time points.

| Experiment | Tank | T0 | T1 | T2 | T3 | OTU |
|---|---|---|---|---|---|---|
| Winter | UV- | 5.9 | 0.1 | | | Unknown OTU2 |
| | treated | | | | | |
| | UV- | 48.8 | 14.3 | 34.2 | 25.2 | Unknown OTU2 |
| | untreated | | | | | |
| Summer | UV- | 47.6 | 1.1 | 7.5 | 22.1 | Unknown OTU2 |
| | untreated | | | | | |
| | UV- | 0.5 | 26.1 | 0.2 | 0.5 | Unknown OTU1 |
| | untreated | | | | | |

Those two main OTU's where unknown at species or genus level in the utilized 16S Silva Database. The sequences of these two OTU's were also checked in green genes and RDP database and no classification were obtained for their taxonomy at both genus and species level. Regarding higher classification of those two OTU's: (i) unknown OTU1 was found to be classified as of the Class Betaproteobacteria, Order Burkholderiales, Family Comamonadaceae, Genus Delftia, and (ii) unknown OTU2 was found to be classified as of the Class Gammaproteobacteria, Order Oceanospirillales, Family Oceanospirillaceae. Genus Profundimonas.

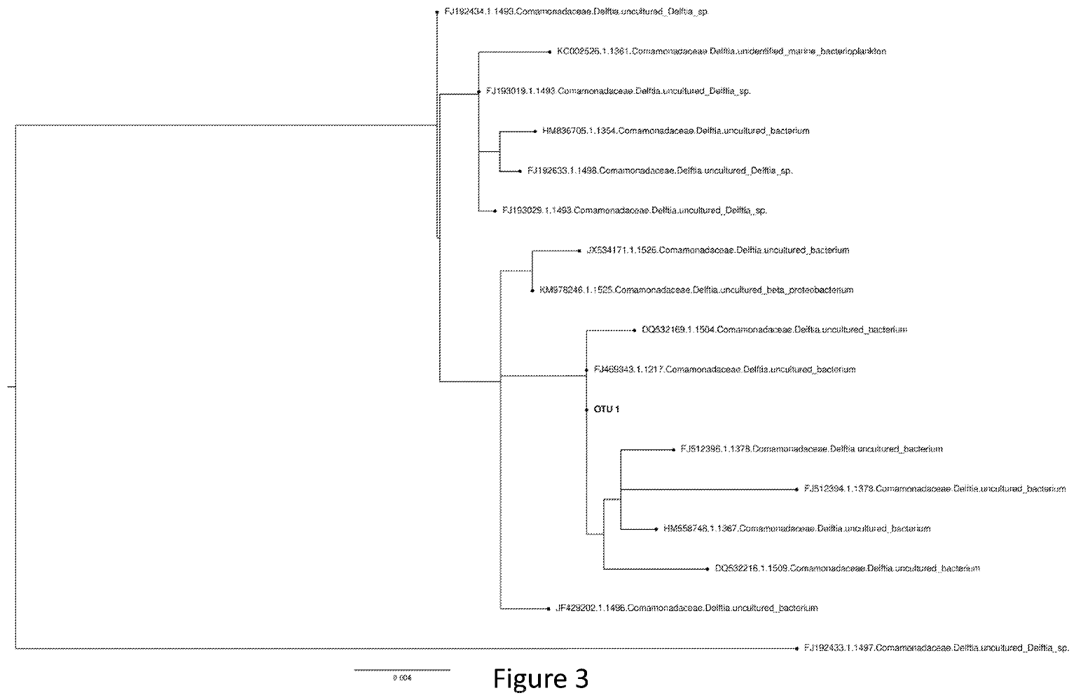

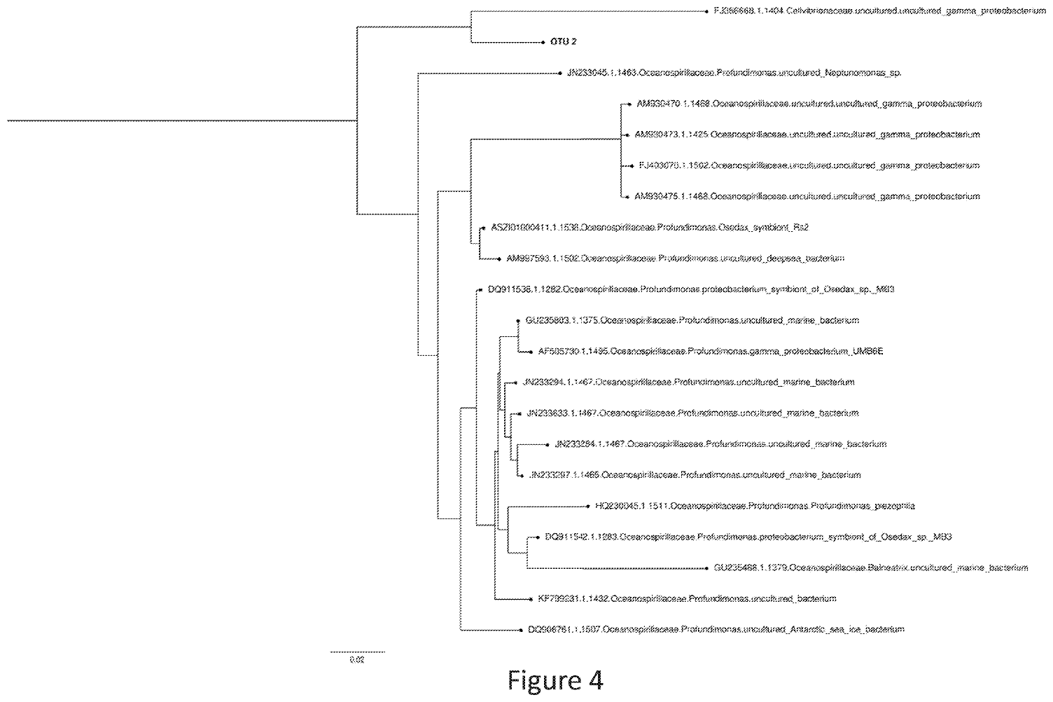

A phylogenetic analysis of those two main OTU's (FIG. 3 and FIG. 4) was further performed. The unknown OTU1 showed a 100% sequence similarity with Delftia uncultured bacterium. However, the other 100% sequence similarity could be misleading, and it also could indicate a new species because of the short sequence length of the 16S to 219 bp. The unknown OTU2 showed a 97.1% sequence similarity with its closest species sequence of genus Profundimonas or uncultured gamma proteobacterium, these 2.9 dissimilarity were noticed in a base substitution at positions 706, 807, 822, 837, 845, 868 and 874.

Certain conclusions may be drawn from the data provided above. It was shown that a large part of the bacterial community in the disease stage (which was associated with Vibrio harveyi, the pathogen causing the disease) are related species that showed competitive growth against the pathogenic species in UV-untreated water, and caused a 40% increase in survival compared to fish grown in UV-treated water. These related bacterial species were only presented when resemblance of 99% sequence similarity was used. Following this, the bacteria were classified using a 99% sequence similarity, into three main bacterial families found in UV-untreated water compared to the fish that are grown in UV-treated water. These bacterial families have a significant effect on fish survival and protection against pathogen.

### Example 2

The following experiment was designed to examine the range of bacterial communities and changes on the skin of Dennis (Sparus aurata) before, during and after controlled infection with Streptococcus iniae, a Gram-positive bacteria, in order to see the interaction between the microbiome and pathogenic bacteria, and compare the results from the previous experiment (using Vibrio harveyi, a Gram-negative bacteria).

The experiment was further designed to test a wider range of microbiome, on another fish species, Barramundi (Lates calcarifer), which is highly sensitive to Streptococcus iniae, in order to examine its skin microbiome in the same way as in EXAMPLE 1 above, to see if there are differences, variety, and/or new bacterial species involved the recovery of a more sensitive fish, compared to Sparus aurata which are less susceptible to this bacteria.

Briefly, 1-gram Barramundi fingerlings were raised up to 60 grams. For the experiment, unvaccinated fish (this species is usually vaccinated at 2 gram against Streptococcus bacteria) were used. Fish which were at least 50 grams are also needed so that they can tagged and handed (taking samples, infection) during the experiment.

After the fish reached a weight of 55 grams, the concentration of Streptococcus iniae was calibrated for a controlled infection by immersion (LD50). The LD50 was found to be 5×10⁷ CFU/ml for one hour immersion after fish were subjected to a handling stress of 5 minutes netting out of water in order to increase sensitivity of fish to the controlled infection.

All fish were tagged on the first day and each fish from each group was sampled throughout the trial several times, a total of 8 samples at different times (time 0, 24, 48, 72 hours, 5 days, one week and one month after the controlled infection with LD50). In addition, water samples were taken from each container at the same time, the water was passed through a 0.2 micron filter and the filter was kept for analysis. All samples were transferred for molecular and bioinformatics analysis.

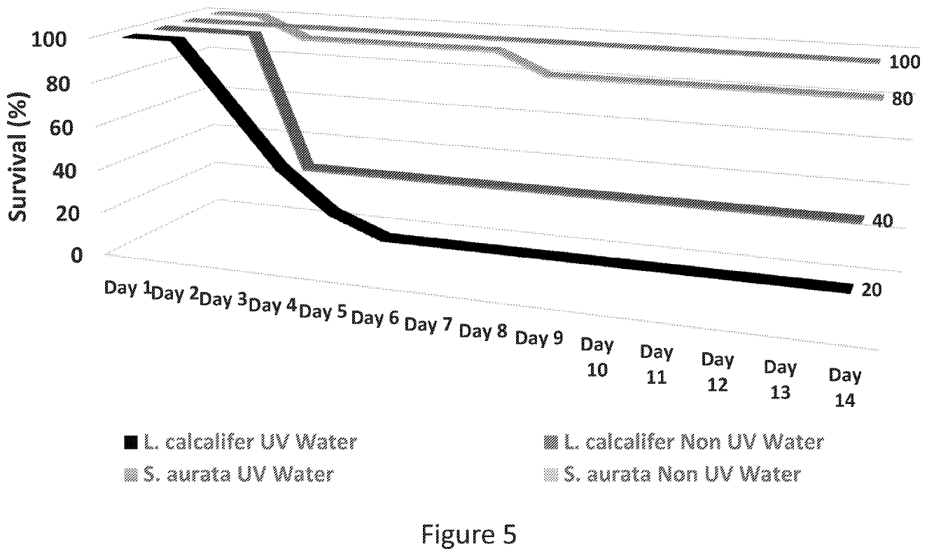

The mortality analysis of the fish in the different groups after infection with the LD50 dose of Streptococcus iniae is shown in FIG. 5.

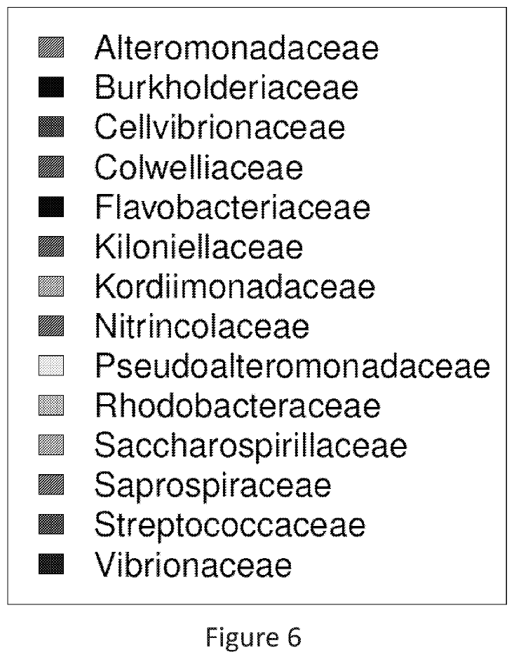

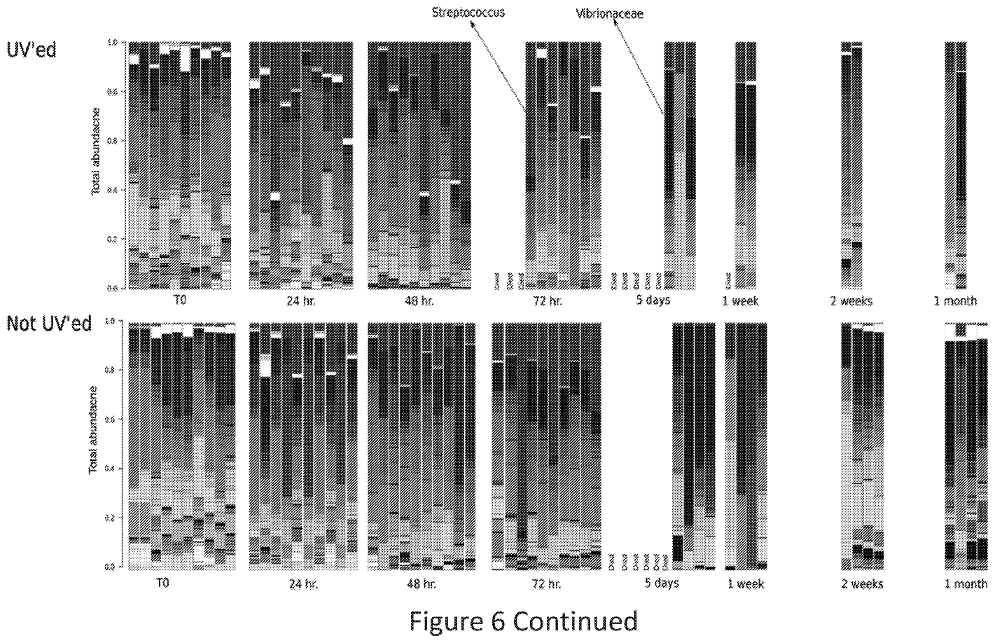

Following the experimental setup, a total of 257 samples were received from each water tank treatment (UV-treated water and UV-untreated water) for both Lates calcarifer and Sparus aurata fish species, each tank containing 10 tagged fish. Swab samples from the later line from fish skin were taken for microbial community analysis. Upon sample collection, the samples were stored in −80 C.° and transferred to DNA extraction using Mobio DNA extraction Kit. Following DNA extraction, the V3-V4 region of 16S rRNA was amplified using the universal primers 515F-806R and illumina sequencing library preparation and samples barcode were performed using Nextera library preparation Kit. Following library preparation, equal molars of samples were mixed and sequenced on Illumina iSeq100 machine. Following sequencing, files (fastq) were curated for quality control, sequences length, chimera and sequencing error using QIIME software, and sequences were clustered using dada2 algorithm and classified using Silva V123 database at 99% sequences similarity. The produced amplicon sequence variant (ASV) table including the abundance of different taxa in each sample were analyzed and bar plots were produced for each treatment (FIG. 6).

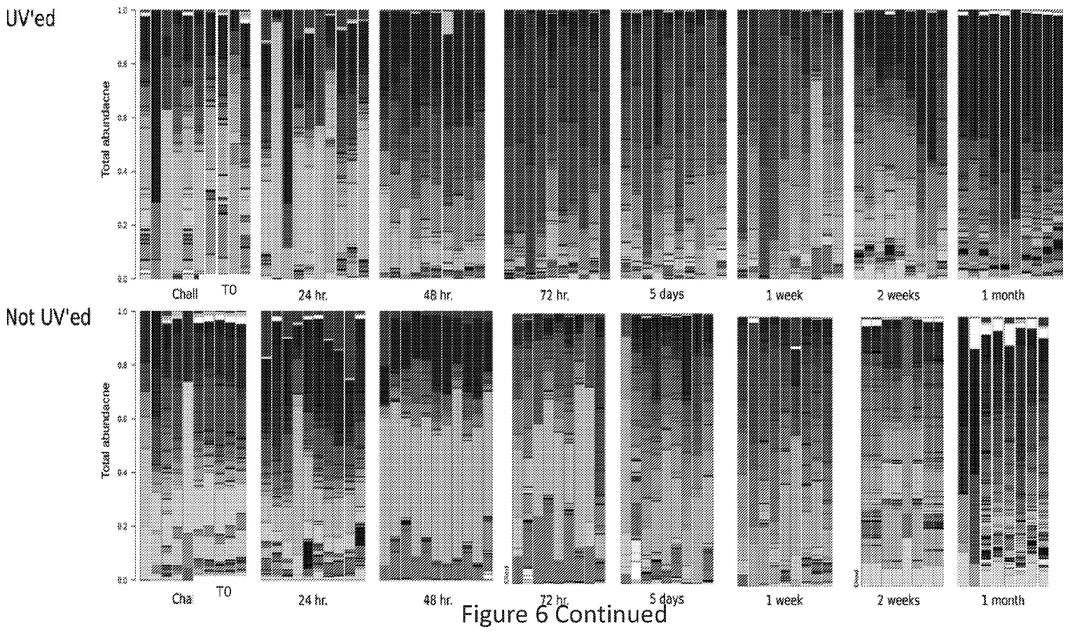

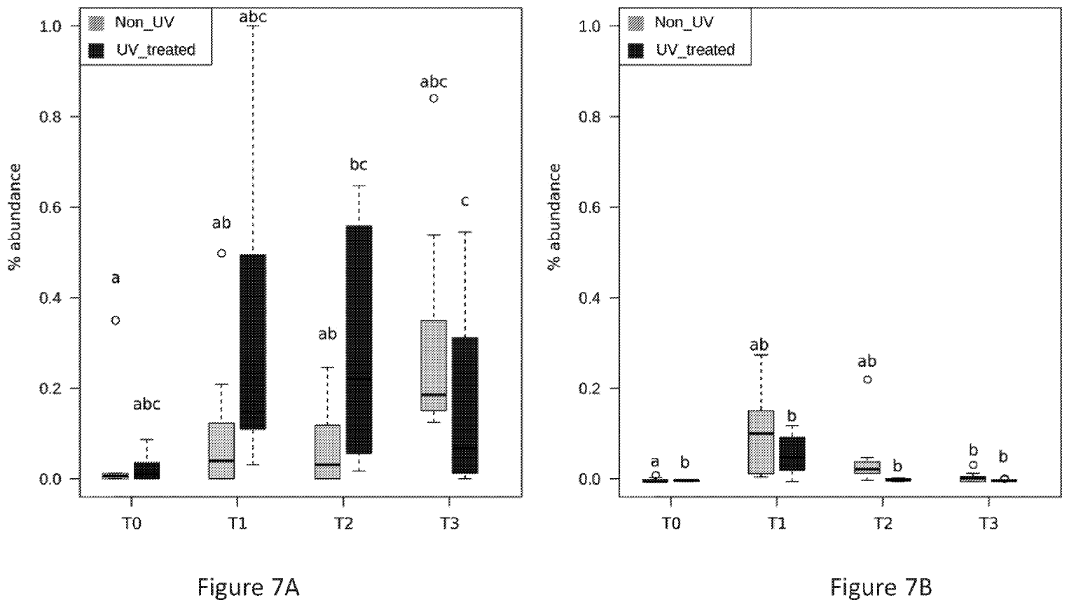

The microbiome analysis clearly shows that Streptococcus iniae pathogen was able to infect Lates calcarifer at higher rate than Sparus aurata fish and causes a higher disease severity, which also lead to higher mortality rates (FIG. 5, FIG. 6). The microbial analysis also shows that Streptococcus iniae was able to infect fish at a significantly higher rate in UV-treated compared to UV-untreated water tanks at 24 and 48 hours after infection for Lates calcarifer (FIG. 7A) while for Sparus aurata there were no significant difference (FIG. 7B).

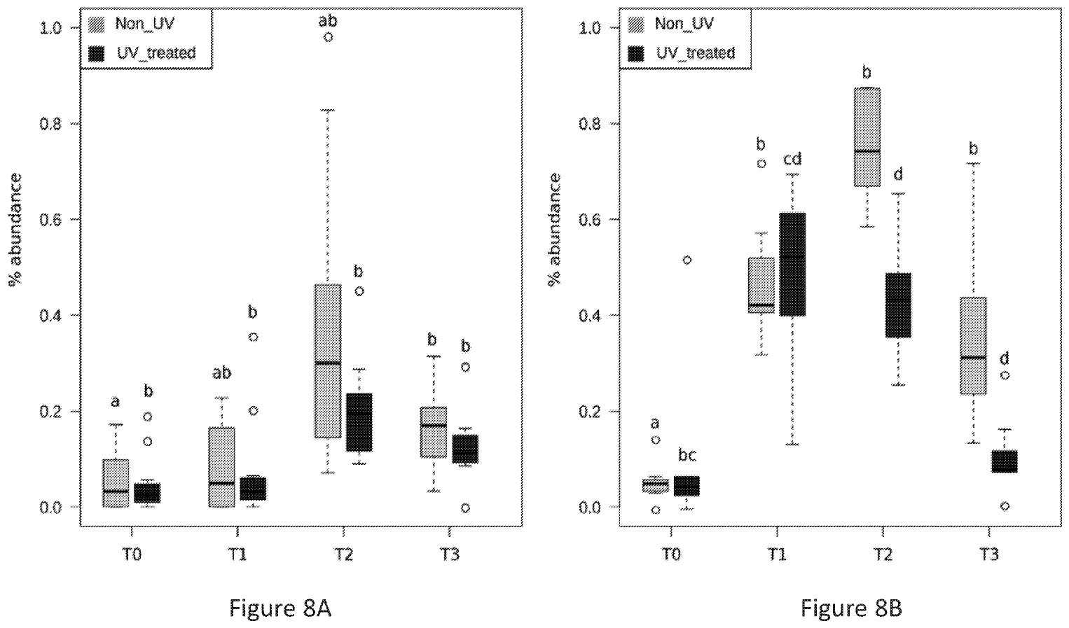

Interestingly, during infection after T0, there is an increase in the abundance of Vibrionaceae family, this increase in abundance was noticed for both fish species at time point T1, T2 and T3 (12, 24 and 72 hour after infection, respectively) with peak abundance at T2, which was also higher in the fish from UV-untreated water tank compared to UV-treated water (FIG. 8).

This Vibrionaceae family was found to consist of two sequences, one belonging to Vibrionaceae Catenococcus genus (OTU3) and the other sequence belonging to an unclassified genus (OTU4) with equal representations.

Those sequences are:

Vibrionaceae Catenococcus (OTU3)
[SEQ ID NO: 7]
GGTGCCAGCAGCCGCGGTAATACGGAGGGTGCGAG
CGTTAATCGGAATTACTGGGCGTAAAGCGCATGCA
GGTGGTTTGTTAAGTCAGATGTGAAAGCCCGGGGC
TCAACCTCGGAATAGCATTTGAAACTGGCAGACTA
GAGTACTGTAGAGGGGGGTAGAATTTCAGGTGTAG
CGGTGAAATGCGTAGAGATCTGAAGGAATACCGGT
GGCGAAGGCGGCCCCCTGGACAGATACTGACACTC
AGATGCGAAAGCGTGGGGAGCAAACAGGATTAGAA
ACCCCTGTAGTCC.
Vibrionaceae unclassified genus (OTU4)
[SEQ ID NO: 8]
GGTGCCAGCAGCCGCGGTAATACGGAGGGTGCGAG
CGTTAATCGGAATTACTGGGCGTAAAGCGCATGCA
GGTGGTTTGTTAAGTCAGATGTGAAAGCCCGGGGC
TCAACCTCGGAATAGCATTTGAAACTGGCAGACTA
GAGTACTGTAGAGGGGGGTAGAATTTCAGGTGTAG
CGGTGAAATGCGTAGAGATCTGAAGGAATACCGGT
GGCGAAGGCGGCCCCCTGGACAGATACTGACACTC
AGATGCGAAAGCGTGGGGAGCAAACAGGATTAGAT
ACCCCTGTAGTCC.

Interestingly, there was an extra sequence (OTU5), which is closely related to OTU2 belonging to Oceanospirillaceae Profundimonas uncultured spp. from the previous experiment. This OTU5, is also noticed to be in significantly abundant during infection representing about 20% of total bacterial communities.

Oceanospirillaceae Profundimonas (OTU5)
[SEQ ID NO: 9]
GGTGCCAGCAGCCGCGGTAATACGGAGGGTGCAAG
CGTTAATCGGAATTACTGGGCGTAAAGCGCGCGTA
GGCGGCCAAGTCAGTCAGATGTGAAAGCCCCGGGC
TTAACCTGGGAACTGCACCTGATACTGCTTGGCTA
GAGTACAGAAGAGGGTGGTGGAATTTCCTGTGTAG
CGGTGAAATGCGTAGATATAGGAAGGAACATCAGT
GGCGAAGGCGGCCACCTGGTCTGATACTGACGCTG
AGGTGCGAAAGCGTGGGGAGCAAACAGGATTAGAA
ACCCCTGTAGTCC.

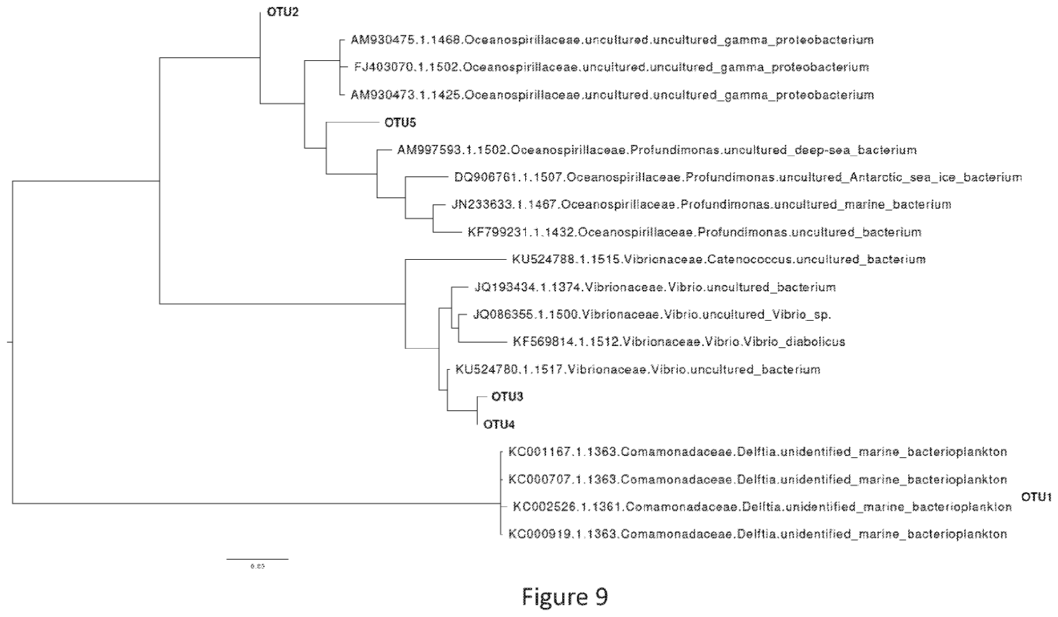

The only difference between OTU3 and OTU4 is one substitution between adenine and thymine. When OTU 3, OTU4 and OTU5 were compared along with the previous sequences OTU2 and OTU1 obtained 99.32, 98.30, 97.10 and 100% respectively with closely related species illustrated in the phylogenetic tree, using Raxml software (FIG. 9).

OTU3 and OTU4 only differ in one nucleotide, however, it is important to mention that these analyses are based on a small fragment of 200 nucleotide bases of the total 16S rRNA subunit.

While this conclusion is important for the development of related probiotics, the results show that related Vibrio species namely Vibrio diabolicus, Vibrio catenococcus and Oceanospirillaceae Profundimonas played an important role in fish survival during Streptococcus iniae infection in both Lates calcarifer and Sparus aurata fish species.

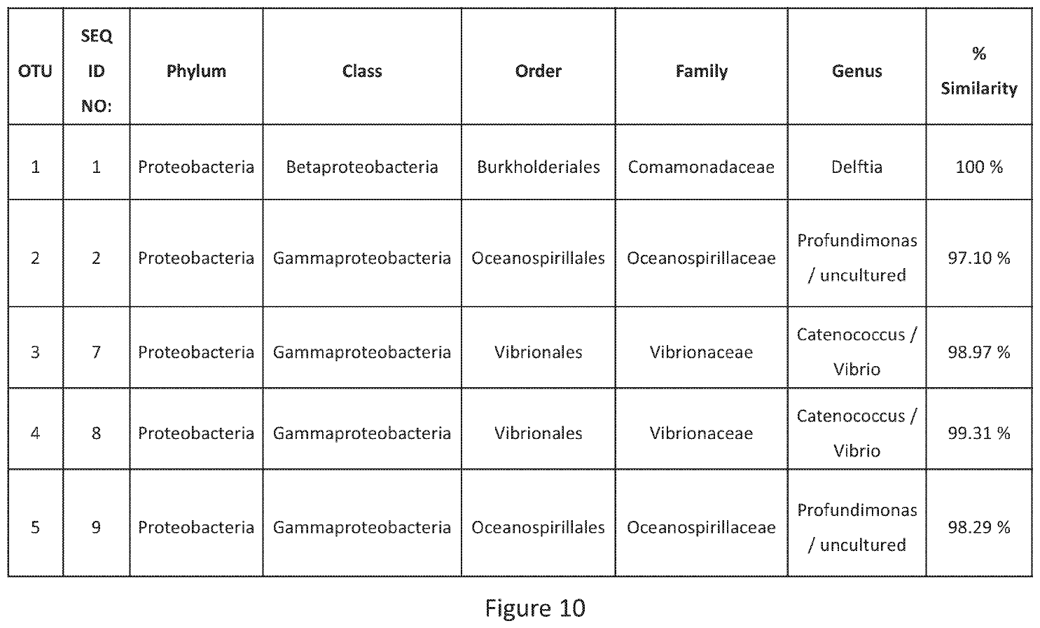

The closely related Oceanospirillales profundiomnas and Comamonadaceae Delftia play an important role during Vibrio harveyi infection in Sparus aurata. Interestingly, at both infections of Streptococcus iniae and Vibrio harveyi in two different experiments, the Oceanospirillales profundiomnas bacterium was abundant during infection and thus it is believed that it has a role in fish survival. FIG. 10 summarizes the OTUs identified herein.

While the present disclosure has been illustrated by description of various embodiments and while those embodiments have been described in considerable detail, there is no intention to restrict or in any way limit the scope of the disclosure to such details. Additional advantages and modifications will readily appear to those skilled in the art. The disclosure in its broader aspects is therefore not limited to the specific details and illustrative examples shown and described. Accordingly, departures may be made from such details without departing from the spirit or scope of the disclosure.

1. A method of predicting the survival of a fish after stress, comprising testing the fish for the presence of bacteria comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9.

2. (canceled)

3. The method of claim 1, wherein the fish is of the Class Actinopterygii.

4. The method of claim 3, wherein the fish is of the Order Perciformes.

5. The method of claim 4, wherein the fish is of the Family Sparidae or Latidae.

6. The method of claim 5, wherein the fish is of the Genus Sparus or Lates.

7. The method of claim 6, wherein the fish is Sparus aurata.

8. (canceled)

9. (canceled)

10. The method of claim 6, wherein the fish is Lates calcarifer.

11. The method of claim 1, wherein the stress is selected from the group consisting of infection with pathogenic bacteria, netting stress, physical injury, and any combination thereof.

12. (canceled)

13. The method of claim 11, wherein the pathogenic bacteria are Gram-negative bacteria.

14. The method of claim 13, wherein the Gram-negative bacteria are selected from the group consisting of the Genus Vibrio, the Genus Pseudomonas, the Genus Edwardsiella, and the Genus Mycobacterium.

15. (canceled)

16. The method of claim 14, wherein the pathogenic bacteria are Vibrio harveyi.

17. The method of claim 11, wherein the pathogenic bacteria are Gram-positive bacteria.

18. The method of claim 17, wherein the Gram-positive bacteria are selected from the group consisting of the Genus Streptococcus, and the Genus Lactococcus.

19. (canceled)

20. The method of claim 18, wherein the pathogenic bacteria are Streptococcus iniae.

21. (canceled)

22. The method of claim 1, wherein the presence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of survival of the fish after the stress

23. (canceled)

24. The method of claim 1, wherein the absence of bacteria comprising a 16S gene comprising the nucleotide sequence set forth in SEQ ID NO: 1, SEQ ID NO: 2, SEQ ID NO: 7, SEQ ID NO: 8, and SEQ ID NO: 9, is predictive of death of the fish after the stress.

25. (canceled)

26. The method of claim 1, further comprising administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

27. (canceled)

28. A method of increasing the survival of a fish after a stress, comprising administering to the fish bacteria comprising a 16S gene comprising the nucleotide sequence set forth in SEQ ID NO: 1, SEQ ID NO: 2, SEQ ID NO: 7, SEQ ID NO: 8, and SEQ ID NO: 9.

29. A composition comprising bacteria comprising a 16S ribosomal RNA gene comprising the nucleotide sequence set forth in SEQ ID NO: 1, SEQ ID NO: 2, SEQ ID NO: 7, SEQ ID NO: 8, or SEQ ID NO: 9.

30. (canceled)

31. The composition of claim 29, for use in a method of increasing the survival of a fish after a stress, comprising administering to the fish bacteria comprising a 16S gene comprising the nucleotide sequence set forth in SEQ ID NO: 1, SEQ ID NO: 2, SEQ ID NO: 7, SEQ ID NO: 8, or SEQ ID NO: 9.

32. (canceled)
